(19) United States
(12) Patent Application Publication
(10) Pub. No.: US 2021/0137377 A1
(43) Pub. Date: May 13, 2021

(54) IN-DIRECT METHOD AND SYSTEM FOR VITAMIN A DEFICIENCY DETECTION

(71) Applicant: Poorn Rajeshbhai MEHTA, Ahmedabad (IN)

(72) Inventor: Poorn Rajeshbhai MEHTA, Ahmedabad (IN)

(21) Appl. No.: 17/045,875

(22) PCT Filed: Dec. 6, 2018
(86) PCT No.: PCT/IN2018/050816

§ 371 Date: Oct. 7, 2020

(30) Foreign Application Priority Data
Apr. 7, 2018 (IN) 201821013340

(51) Int. Cl. A61B 3/06; A61B 3/02; G01N 33/82

(52) U.S. Cl. CPC A61B 3/063; G01N 33/82; A61B 3/022

(57) ABSTRACT

The present invention relates to indirect diagnosis of deficiency of Vitamin A, without taking blood sample. The instrument combines two simple principles for full functionality, An individual having Vitamin A deficiency can be differentiated from a healthy person on 2 basis: (1) The ability to clearly identify pictorial representation of an object in the environment having low amount of light intensity; and (2) The time being taken for eyes to adapt to a significantly different lighting situation. The standardization of the instrument has to be done in nearby area in relatively healthy population having rich diet in vitamin A or it is to be adopted based on findings of other area, The diagnosis can be confirmed after repeating the same set of tests, after giving vitamin A in appropriate dosages, and allowing enough time to pass for that dosage to take an effect (generally, 1 to 2 weeks).

### FIELD OF THE INVENTION

The present invention relates to indirect diagnosis of deficiency of Vitamin A, without taking blood sample.

### BACKGROUND OF THE INVENTION

Vitamin A is very important nutrient for functions of various parts of the body. Skin integration & mucous membrane integration are dependent on Vitamin A level in Blood. Vitamin A has also important role in vision according to the medical science. The vision problems are due to defective rhodopsin production in retina in case of Vitamin A deficiency. A person with Vitamin A deficiency takes significantly longer time for light adaptation as well as, facing difficulty in identifying objects in dimly lit environment. Particularly vehicular accidents will be more at night by drivers having vitamin A deficiency. This happens due to the delay in adaptation in relative darkness which is created due to sudden glare of light. It is difficult to notice night blindness by most of the people as mostly there are artificial light sources present in close proximity, such as electric bulb. Where there is no electricity—mostly too remote areas—people are so habituated with existing deficiency of Vitamin A that they take it as granted for normal.

Nearly 30,000 children become blind due to Vitamin-A deficiency every year. Also it is common in pregnant women resulting in low birth weight babies & premature deliveries. In addition to blindness due to keratomalacia & vision problems Vitamin A deficiency leads to various devastating effect on body by weakening of epithelial linings. This includes susceptibility to various infections like respiratory tract infection (Cough, cold, asthma) including swine flu, diarrhea, boils, urinary tract infection, ear infection, conjunctivitis, menstrual problems, sterility, failure to gain weight, skin infections, increase in infant mortality, sexual transmitted diseases, HIV etc. It also leads to problems of circulatory system, alimentary system, immune system and bone marrow.

This shows importance of detection and prevention of Vitamin A deficiency. However, current methods of testing for Vitamin A deficiency include complex procedure, require blood samples, and quite costly. Therefore, in the present invention, novel method and system has been developed for the detection of Vitamin-A deficiency; which is very cost effective and simple in procedure. Moreover, this invention does not require any blood test, while taking only a few minutes to complete a test. Dark adaptometry testing is useful in the diagnosis and management of vitamin-A deficiency, retinal degenerations, senile miosis, high myopia and other night blinding conditions. Along with this, the device will also test the intensity of light required to visualize the pictures inside the instrument, which will suggest night blindness and indirect indication of vitamin A deficiency or presence of other conditions. Measurement of vitamin A level is essential not only in hospitals but also in few departments like road transport service and railway service employments to prevent accidents related to vision defect.

### OBJECT OF THE INVENTION

The main object of the present invention is to develop a portable, affordable, and standardized instrument—which can give close estimation about the presence of Vitamin A deficiency in an individual. Also, it has to bring the cost of test to a minimal figure, and to take little time for the testing and analysis.

Further, the invention has to refrain from taking blood samples in order to get the test done. This invention does not require any sample at all to conduct the test.

The another object of the present innovation is to provide the feasibility and flexibility in such a manner, that it is able to conduct tests in remote areas, villages, and at places where even electricity is not present. For this, the invention is designed to be able to run by using the commercial power banks.

One more objective is that making this device rugged, and suitable for longer lifetime. That is why, this device uses high end technology such as Electronic Paper.

### STATEMENT OF INVENTION

Development of an instrument for measuring (a) the required intensity of light to visualize the object clearly, and (b) time required to adapt to the sudden light change; based on which, an indirect prediction of detection of Vitamin A deficiency and to allied conditions can be done.

The instrument combines two simple principles for full functionality. An individual having Vitamin A deficiency can be differentiated from a healthy person on 2 basis: (1) The ability to clearly identify pictorial representation of an object in the environment having low amount of light intensity; and (2) The time being taken for eyes to adapt to a significantly different lighting situation. The standardization of the instrument has to be done in nearby area in relatively healthy population having rich diet in vitamin A or it is to be adopted based on findings of other area. The diagnosis can be confirmed after repeating the same set of tests, after giving vitamin A in appropriate dosages, and allowing enough time to pass for that dosage to take an effect (generally, 1 to 2 weeks).

### BRIEF DESCRIPTION OF THE DRAWINGS

It will be convenient to further describe the present invention with respect to the accompanying drawings that illustrate possible arrangements of the invention. Other arrangements of the invention are possible, and consequently the particularity of the accompanying drawings is not intended to be limiting of the present invention.

FIG. 1 flow chart of he present invented system

### DETAILED DESCRIPTION OF THE INVENTION

Before explaining the present invention in detail, it is to be understood that the invention is not limited in its application to the details of the construction and arrangement of parts illustrated in the accompanying drawings. The invention is capable of other embodiments, as depicted in different figures as described above and of being practiced or carried out in a variety of ways. It is to be understood that the phraseology and terminology employed herein is for the purpose of description and not of limitation.

As shown in FIG. 1 At the boot up, the present invention device initiates all the hardware one by one. Upon success, it enters the software initialization stage. Configuration file will be read from the MicroSD card and using the data written in it, variables of the program will be initiated.

Brightness control is a function that is responsible for driving the White LED which is facing the E Paper, while taking into account feedback from PIN photodiode, and command received from Central Test Algorithm. Also, the brightness value in real time will be calculated and given to LCD to display, by this function.

Image control is a function that is responsible for displaying images from a selected pool of images, changing the image on command of Central Test Algorithm, and also to change it in the way defined by the user. It also communicates with the E Paper module and upon detection of an error signal, it will send a message to LCD. Also, if a specific pool of images/a single image is not found, then it will get back to the central test algorithm with specifics.

Software timer is set of 16 bit timers, used for calculating the time required for a particular task by user, on the instruction of Central Test Algorithm.

Central Test Algorithm is the heart of the device. It is designed using known data and information about the method of differentiating a healthy person from a person having Vitamin A deficiency, by the means of measuring and comparing the abilities to adapt to low light/complete dark/either of that after an intense flash, and also—the ability to view the displayed images clearly in very low light.

To improve the invention further, MicroSD card is used. It will keep the record of all the tests conducted and the user will send the data stored in the SD card to the developer. This will create a database, eventually large enough to accurately determine range of intensity and time required for dark adaptation. Also, the ranges will be divided as per the age groups and gender, also present in test data. This will enable the invention to be used widely, with high reliability and low limitations.

To use the present invented device, first to detect the defect in eye sight, in the present invention, instrument is created, which has a controlled environment in terms of managing visible light intensity within an instrument. It also has a timer, which calculates the time required by an individual with very high accuracy. With the help of microcontroller and special programming, a circuit has been developed which is able to return extremely precise results over a large period of time. The consisting parts of the present invented instrument components are very durable and are having exceptionally long life span, such as Power LED, LCD display, Electronic Paper Module, AUR 8 bit microcontroller, MicroSD Card, PIN Photodiode (as light sensor) etc. The brightness of power LED—responsible for all the lighting inside the machine, is easily, accurately and precisely controlled in the range from Zero Lux to Thousands of Lux. PIN Photodiode has been included to provide a feedback, which ensures reliable performance even after years of continuous usage. The device is designed in a way that it can offer the functionality of a semi-autonomous testing mode, which only requires input from user on specific instances—upon reaching specific checkpoints in the test. In this mode, variation of light intensity, changing of the image on Electronic Paper, calculating the time required in seconds for dark adaption, etc. will be automated. The present invention system includes the principle of “dark adaptation” of human eye, which means that “the adjustment of the eye to low light intensities”.

By using the present invention system, If the evaluation for this instrument will be measured by assessing around “N” number of persons in specified community and record will be maintained. Those who require intensity of light more than X units as per this present invented system will be given Vitamin A “Y” units.

They will be tested again for adaptation with the same instrument after one week and will compare the improvement, once the result of this system are recorded. Every group of individuals with specified range of intensity of light required for viewing image (in lux) and the time required to adapt to the darker scene (in seconds) will be given a fixed low dosage of Vitamin A. After an interval of a week, they will be tested again, and the results will be compared with past records. This will give accurate description of the scenario, and eliminate or confirm the presence of Vitamin A deficiency.

The serum vitamin A level of significant number of individuals in different categories will be carried out if this becomes feasible (by blood test method). In that case correlation between readings of instrument and serum Vitamin A level will be measured and base of results date if Normograph based on regression equation. Which is helpful to know the deficiency vitamin A level based on instrument reading in future.

### SUMMARY OF THE INVENTION

The present invention is consists with electronic paper module, LCD to display test object, MicroSD card and key pad

Wherein electronic paper is replaced by LCD to display multiple images because n the electronic paper the latter one uses technique for displaying pictures which is natural. In everyday life, humans ‘see’ objects by capturing and processing the visible light that is reflected from the object(s) in sight. LCD uses a different technique—by emitting its own light. However, Electronic Paper does not have any light source and work on the principle of light reflection.

Wherein a (i.e. 4×20 Size) LCD is used to display test data, recorded values, and useful information. It forms a part of User Interfacing by giving proper output.

Wherein MicroSD card used in the system to provide storage memory for completed tests, and to store configuration file which can be easily updated. One more SD card will lie within the Electronic Paper module for storing the images. As it does not interact with the system directly, it is not shown in the block diagram.

Wherein (4×4 Size) Keypad is used to take inputs from user. It is the best match for this application, as it is space efficient, and providing all necessary functionalities.

White Light Emitting Diode is used to produce controlled amount of illumination in the device—on the Electronic Paper. The intensity is going to be controlled via Pulse Width Modulation technique. The LED will be driven directly from microcontroller digital output pin. Another similar component is place with different orientation in the device to add the functionality of a variant of Dark Adaption test.

PIN Photodiode is used to measure the light intensity. The sensor will be placed, very next to the E-Paper display. Using the feedback value given to the microcontroller from this sensor, LED will be controlled. As PIN Photodiodes have very long life spans along with high accuracy and precision, the device will not need any kind of maintenance—at least for the part of controlling the light intensity. The values of photodiode output, at known PWM values of LED will be correlated and standardized using High End Lux Meter.

The present invented device is not use to measure accurate Vitamin A levels in an individual's blood. Rather, it works on process of elimination, and empirical proof of Vitamin A deficiency.

The test will be conducted in two parts, with a margin of approximately one week being in between. After completion of first part, the estimated chances of a person having Vitamin A deficiency can be calculated. For this, verified data of individuals—performing under standardized blood test, and performing under this method, will be used. Thus, larger the dataset, more accuracy obtained in results post first part completion. However, the result of second part is not dependent in such manner. After first part, if an individual is found to be on the higher probability side of Vitamin A deficiency—a small dosage of Vitamin A will be given to that individual. After allowing several days to pass, in order to Vitamin A dosage to take effect, second part of the test will be conducted. If any significant improvements are observed in this stage then, it can be stated that the individual was indeed suffering from Vitamin A deficiency. Post this, further dosage (if required) can be calculated based on previous observations.

The test will be in the form semi-automated sequences of steps and recording of values. There will be options to modify the sequence in certain manners.

Main component of the test is to measure response time of the individual under test, and also to measure the intensity of light at which, the individual is able to perceive images displayed on Electronic Paper clearly. Response time can also be measured after exposing the individual to short lasting pulse of bright light, and then observing the amount of time needed to regain proper vision in low light.

Described above, only the preferred embodiment of the present invention only, not for the present invention any formal restrictions, although the invention has been revealed as the preferred embodiment, however, not intended to limit the present invention, any skilled in the art technical staff, without departing from the scope of the present invention within the program, can be used when the contents of the above disclosed techniques make little changes or substitutions and changes in the equivalent embodiments, but any aspect of the invention without departing from the content, according to the present invention, technical substance of any of the above embodiments when taken in a simple modification, equivalent variation and modification as would fall within the scope of the present invention the technical solution.

I claim:

1. In-Direct method and system for Vitamin A deficiency detection consists with electronic paper module, LCD to display test object, MicroSD card, key pad, LED
Wherein electronic paper is provided to view the letters and pictures in a natural manner, as it does not have any light source and rather, work on the principle of light reflection;
Wherein a LCD is provided to display test data, recorded values, and useful information and it also serves the purpose of user interfacing by giving proper output;
Wherein MicroSD card used in the system to provide storage memory for completed tests, and to store configuration file which can be easily updated;
Wherein Keypad is provided to take inputs from user.
Wherein White Light Emitting Diode is used to produce controlled amount of illumination in the device—on the Electronic Paper, and another similar component used to provide flash light, as a first stage of dark adaption test;
Wherein PIN Photodiode is used to measure the light intensity.

2. In-Direct method and system for Vitamin A deficiency detection as claimed in claim 1 wherein multiple number of MicroSD cards are provided, one within the Electronic Paper module for storing the images.

3. In-Direct method and system for Vitamin A deficiency detection as claimed in claim 1 wherein the intensity of light is going to be controlled via Pulse Width Modulation technique.

4. In-Direct method and system for Vitamin A deficiency detection as claimed in claim 1 wherein the LED is be driven directly from microcontroller digital output pin.

5. In-Direct method and system for Vitamin A deficiency detection as claimed in claim 1 wherein the sensor is be placed, very next to the E-Paper display.

6. In-Direct method and system for Vitamin A deficiency detection as claimed in claim 1 wherein the feedback value given to the microcontroller from this sensor, LED will be controlled.

7. In-Direct method and system for Vitamin A deficiency detection as claimed in claim 1 wherein the values of photodiode output, at known PWM values of LED will be correlated and standardized using High End Lux Meter.

8. In-Direct method and system for Vitamin A deficiency detection as claimed in claim 1 wherein to microcontroller with programmed is provided to creates the controlled environment in terms of managing visible light intensity within an instrument.
